(19) United States
(12) Patent Application Publication
(10) Pub. No.: US 2022/0125879 A1
(43) Pub. Date: Apr. 28, 2022

(54) ALBUMIN-BOUND COMPOSITION INCLUDING LRRD2 OF SLIT3 PROTEIN FOR PREVENTION OR TREATMENT OF BONE-RELATED DISEASES

(71) Applicant: Daewoong Pharmaceutical Co., Ltd., Gyeonggi-do (KR)

(72) Inventors: Jung Min KOH, Seoul (KR); Sung Sub KIM, Daejeon (KR); Kyong Hoon AHN, Seoul (KR)

(73) Assignee: Daewoong Pharmaceutical Co., Ltd., Gyeonggi-do (KR)

(21) Appl. No.: 17/434,389

(22) PCT Filed: Feb. 27, 2020
(86) PCT No.: PCT/KR2020/002825

§ 371 Date: Aug. 26, 2021

(30) Foreign Application Priority Data
Feb. 27, 2019 (KR) 10-2019-0023376

(51) Int. Cl. A61K 38/17; A61K 47/64; A61P 19/10

(52) U.S. Cl. CPC A61K 38/1709; A61P 19/10; A61K 47/643

(57) ABSTRACT

The present invention relates to a composition comprising albumin-bound LRRD2 of the SLIT3 protein for prevention or treatment of bone-related diseases.

### TECHNICAL FIELD

The present invention relates to a composition comprising albumin-bound LRRD2 of the Slit3 protein for prevention or treatment of bone-related diseases.

### BACKGROUND ART

Slit proteins are well-known proteins that regulate the movement of neurons and axons during the developmental process of the nervous system. It is known that a Slit protein can act with a Robo receptor to regulate physiological activity, and serves as a factor that regulates various intracellular processes in various tissues such as heart, lung, kidney, and breast tissues, and as it has been recently reported that Slit proteins play an important role in the regulation of growth, adhesion ability, and migration ability of cells, it was reported that Slit proteins can participate in the migration in the differentiation of cells and the occurrence and metastasis of cancer.

Based on such a background, the present inventors have revealed that LRRD2 of Slit3 can be usefully used as a composition for prevention or treatment of fractures or osteoporosis and a biomarker for predicting the risk of outbreak of fractures or osteoporosis because LRRD2 of Slit3 increases bone formation, reduces bone resorption, and has a negative correlation with the incidence of osteoporosis in cell and animal models (Korean Patent No. 10-1617497). Since LRRD2 needs to be administered as an injection, patients need to visit the hospital, but LRRD2 has a very short in vivo half-life, so its administration cycle should be shortened in order to exhibit the medicinal effects thereof, and it is expected that a problem in that its efficacy is reduced due to the associated excessive use of the drug occurs.

Thus, the present inventors have developed an HSA-Slit3 LRRD2 fusion protein with improved efficacy by increasing the in vivo half-life of LRRD2, thereby completing the present invention.

### DISCLOSURE

### Technical Problem

An object of the present invention is to provide a composition in which the efficacy of LRRD2 of the Slit3 protein for preventing or treating bone-related diseases has been improved.

### Technical Solution

To achieve the above-described object, the present invention provides a pharmaceutical composition comprising albumin-bound LRRD2 of the Slit3 protein for prevention or treatment of bone-related diseases.

According to a preferred exemplary embodiment of the present invention, the albumin may be human serum albumin.

According to another preferred exemplary embodiment of the present invention, the human serum albumin may be bound to the N-terminus of LRRD2 of the Slit3 protein.

According to still another preferred exemplary embodiment of the present invention, the human serum albumin may include an amino acid sequence of SEQ ID NO: 2.

According to yet another preferred exemplary embodiment of the present invention, the LRRD2 of the Slit3 protein may include an amino acid sequence of SEQ ID NO: 3.

According to yet another preferred exemplary embodiment of the present invention, the pharmaceutical composition may further include a linker between the albumin and the LRRD2 of the Slit3 protein.

According to yet another preferred exemplary embodiment of the present invention, the linker may be (GGGGS)n (SEQ ID NO: 5), wherein n may be an integer from 1 to 10.

According to yet another preferred exemplary embodiment of the present invention, the pharmaceutical composition may be administered as an injection.

According to yet another preferred exemplary embodiment of the present invention, the bone-related disease may be any one or more selected from the group consisting of osteoporosis, fractures, bone loss, osteoarthritis, metastatic bone cancer, and Paget's disease.

### Advantageous Effects

Since albumin-bound LRRD2 of the Slit3 protein exhibits the same cytological efficacy as albumin-unbound LRRD2 of the Slit3 protein and has a significantly increased in vivo half-life compared to albumin-unbound LRRD2 of the Slit3 protein, bone-related diseases can be more effectively prevented or treated.

### DESCRIPTION OF DRAWINGS

FIG. 1 illustrates a composition of a fusion protein in which an albumin of the present invention is bound to the N-terminus of Slit3 LRRD2 and an amino acid sequence thereof.

FIG. 2 illustrates the results of performing SDS-PAGE after isolating and purifying an SP cystatin S-HSA-Slit3LRRD2 fusion protein.

FIG. 3 graphically illustrates the receptor binding ability of various forms of HSA-Slit3 LRRD2 fusion proteins.

FIG. 4 illustrates the results of confirming the migration ability of osteoblasts according to the treatment of various forms of HSA-Slit3 LRRD2 fusion proteins.

FIG. 5 illustrates the results of confirming the differentiation ability of osteoclasts according to the treatment of various forms of HSA-Slit3 LRRD2 fusion proteins.

FIG. 6 illustrates the results of confirming the results of confirming (A) the migration ability of osteoblasts, (B) the β-catenin activity of osteoblasts, and (C) the differentiation ability of osteoclasts, according to the treatment of the HSA-Slit3 LRRD2 fusion protein.

FIG. 7 illustrates the plasma concentration-time profiles of Slit3 LRRD2 after IV administration of Slit3 LRRD2 (≡, “Slit3”) and HSA-Slit3 LRRD2 (▪, “HSA-Slit3”) to fasted male ICR mice.

### MODES OF THE INVENTION

As described above, LRRD2 of the Slit3 protein may be used for prevention or treatment of fractures or osteoporosis by increasing bone formation and reducing bone resorption, but LRRD2 has a very short in vivo half-life, so its administration cycle should be shortened in order to exhibit the medicinal effects thereof, and it is expected that a problem in that its efficacy is reduced due to the associated excessive use of the drug occurs

Thus, the present inventors have sought a solution to the above-described problem by enhancing the in vivo half-life of LRRD2 to develop an HSA-Slit3 LRRD2 fusion protein with improved efficacy. Since albumin-bound LRRD2 of the Slit3 protein exhibits the same cytological efficacy as albumin-unbound LRRD2 of the Slit3 protein and has a significantly increased in vivo half-life compared to albumin-unbound LRRD2 of the Slit3 protein, bone-related diseases can be more effectively prevented or treated.

Hereinafter, the present invention will be described in more detail.

The present invention provides a composition comprising albumin-bound LRRD2 of the SLIT3 protein for prevention or treatment of bone-related diseases.

In the pharmaceutical composition of the present invention, the “LRRD2 of the Slit3 protein” refers to a second leucine-rich repeat domain (LRRD2) in the Slit3 protein.

As used herein, the term “Slit3 LRRD2” refers to “LRRD2 of the Slit3 protein” and may be used interchangeably.

In the pharmaceutical composition of the present invention, the albumin may be human serum albumin, rhesus serum albumin (RhSA), cynomolgus monkey serum albumin (CySA), or murine serum albumin (MuSA), and preferably human serum albumin. The Slit3 LRRD2 has an in vivo half-life of Slit3 LRRD2 in the presence of human serum albumin that is at least 10-fold longer than that of Slit3 LRRD2 in the absence of human serum albumin.

In a specific exemplary embodiment of the present invention, the serum half-life of Slit3 LRRD2 in the presence of human serum albumin is 14-fold longer than that of Slit3 LRRD2 in the absence of human serum albumin.

In the pharmaceutical composition of the present invention, the human serum albumin and Slit3 LRRD2 may be bound in the order of the human serum albumin and Slit3 LRRD2, or vice versa. Preferably, the human serum albumin and Slit3 LRRD2 are bound in this order. For example, when the human serum albumin binds to the N-terminus of Slit3 LRRD2, Slit3 LRRD2 has the best in vivo half-life and therapeutic efficacy for bone-related diseases, and when the human serum albumin binds to the C-terminus thereof, it is possible to exhibit an effective efficacy even though there may be a difference in degree.

In the pharmaceutical composition of the present invention, as the human serum albumin, a full-length amino acid sequence consisting of 609 amino acids or a fragment comprising a partial amino acid sequence thereof may be used. The full-length amino acid sequence of the human serum albumin is disclosed in the NCBI GenBank: AAA98797.1, and in an exemplary embodiment of the present invention, the form of a fragment consisting of the 25th to 609th amino acids (585 amino acids) from the full-length human serum albumin consisting of 609 amino acids was used. In the pharmaceutical composition of the present invention, the human serum albumin consists of the following SEQ ID NO: 2:

(SEQ ID NO: 2)
DAHKSEVAHRFKDLGEENFKALVLIAFAQYLQQCP
FEDHVKLVNEVTEFAKTCVADESAENCDKSLHTLF
GDKLCTVATLRETYGEMADCCAKQEPERNECFLQH
KDDNPNLPRLVRPEVDVMCTAFHDNEETFLKKYLY
EIARRHPYFYAPELLFFAKRYKAAFTECCQAADKA
ACLLPKLDELRDEGKASSAKQRLKCASLQKFGERA
FKAWAVARLSQRFPKAEFAEVSKLVTDLTKVHTEC
CHGDLLECADDRADLAKYICENQDSISSKLKECCE
KPLLEKSHCIAEVENDEMPADLPSLAADFVESKDV
CKNYAEAKDVFLGMFLYEYARRHPDYSVVLLLRLA
KTYETTLEKCCAAADPHECYAKVFDEFKPLVEEPQ
NLIKQNCELFEQLGEYKFQNALLVRYTKKVPQVST
PTLVEVSRNLGKVGSKCCKHPEAKRMPCAEDYLSV
VLNQLCVLHEKTPVSDRVTKCCTESLVNRRPCFSA
LEVDETYVPKEFNAETFTFHADICTLSEKERQIKK
QTALVELVKHKPKATKEQLKAVMDDFAAFVEKCCK
ADDKETCFAEEGKKLVAASQAALGL.

In the pharmaceutical composition of the present invention, the Slit3 LRRD2 is human-derived, and a full-length amino acid sequence of LRRD2 in the Slit3 protein consisting of 1523 amino acids or a fragment comprising a partial amino acid sequence thereof may be used. The full-length amino acid sequence of the Slit3 protein is disclosed in the NCBI GenBank: AAQ89243.1, and in an exemplary embodiment of the present invention, as Slit3 LRRD2, the form of a fragment consisting of the 278th to 486th amino acids (209 amino acids) from the full-length Slit3 protein consisting of 1523 amino acids was used. In the pharmaceutical composition of the present invention, Slit3 LRRD2 consists of an amino acid sequence of the following SEQ ID NO: 3:

(SEQ ID NO: 3)
ISCPSPCTCSNNIVDCRGKGLMEIPANLPEGIVEI
RLEQNSIKAIPAGAFTQYKKLKRIDISKNQISDIA
PDAFQGLKSLTSLVLYGNKITEIAKGLFDGLVSLQ
LLLLNANKINCLRVNTFQDLQNLNLLSLYDNKLQT
ISKGLFAPLQSIQTLHLAQNPFVCDCHLKWLADYL
QDNPIETSGARCSSPRRLANKRISQIKSKKFRCS.

In the pharmaceutical composition of the present invention, “Slit3 LRRD2” may include a functional equivalent of the amino acid sequence of SEQ ID NO: 3.

The “functional equivalent” has a sequence homology of at least 70% or more, preferably 80% or more, more preferably 90% or more, and even more preferably 95% or more with the amino acid sequences of SEQ ID NOS: 1 to 4 of the present invention by the addition, substitution, or deletion of amino acids of a protein or peptide, and refers to a protein or peptide exhibiting physiological activity substantially equivalent to that of a protein or peptide consisting of amino acid sequences of SEQ ID NOS: 1 to 4.

Specifically, for the fusion protein included in the pharmaceutical composition of the present invention, not only a protein or peptide having a wild-type amino acid sequence thereof, but also an amino acid sequence variant thereof may also be included in the scope of the present invention. The amino acid sequence variant refers to a protein or peptide having a sequence different from a wild-type amino acid sequence of Slit3 LRRD2 by deletion, insertion, non-conservative or conservative substitution of one or more amino acid residues, or a combination thereof.

Amino acid exchanges possible in proteins and peptides that do not entirely change the activities of the molecules are known in the art (H. Neurath, R. L. Hill, The Proteins, Academic Press, New York, 1979). The most typically occurring exchanges are exchanges between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. In some cases, amino acids may also be modified by phosphorylation, sulfation, acetylation, glycosylation, methylation, famesylation, or the like.

The Slit3 LRRD2 of the present invention, or variants thereof can be extracted from nature or synthesized (Merrifleld, J. Amer. Chem. Soc. 85:2149-2156, 1963) or prepared by a gene recombinant method based on a DNA sequence (Sambrook et al., Molecular Cloning, Cold Spring Harbour Laboratory Press, New York, USA, 2d Ed., 1989).

In the pharmaceutical composition of the present invention, a linker may be further included between albumin and LRRD2 of the Slit3 protein. A preferred linker type may be (GGGS)n (SEQ ID NO: 5), wherein n may be an integer from 1 to 10, and preferably n may be an integer from 1 to 5.

The Slit3 LRRD2 of the present invention may be subjected not only to albumin fusion, but also to fusion or PEGylation of an Fc protein of IgG, and the like in order to enhance the in vivo half-life thereof.

The pharmaceutical composition of the present invention may be in the form of various oral or parenteral formulations. When the pharmaceutical composition is formulated, the composition may be prepared by using a buffer (for example, a saline solution or PBS), an antioxidant, a bacteriostatic agent, a chelating agent (for example, EDTA or glutathione), a filler, an extender, a binder, an adjuvant (for example, aluminum hydroxide), a suspension agent, a thickener, a wetting agent, a disintegrant, or a surfactant, a diluent or an excipient.

Examples of a solid preparation for oral administration include a tablet, a pill, a powder, granules, a capsule, and the like, and the solid preparation is prepared by mixing one or more compounds with one or more excipients, for example, starch (including corn starch, wheat starch, rice starch, potato starch, and the like), calcium carbonate, sucrose, lactose, dextrose, sorbitol, mannitol, xylitol, erythritol maltitol, cellulose, methyl cellulose, sodium carboxymethylcellulose and hydroxypropymethyl-cellulose, gelatin, or the like. For example, a tablet or a sugar tablet may be obtained by blending an active ingredient with a solid excipient, pulverizing the resulting blend, adding a suitable auxiliary agent thereto, and then processing the resulting mixture into a granular mixture.

Further, in addition to simple excipients, lubricants such as magnesium stearate and talc are also used. A liquid preparation for oral administration corresponds to a suspension agent, a liquid for internal use, an emulsion, a syrup, and the like, and the liquid preparation may include, in addition to water and liquid paraffin which are simple commonly used diluents, various excipients, for example, a wetting agent, a sweetener, an odorant, a preservative, and the like. In addition, in some cases, cross-linked polyvinyl pyrrolidone, agar, alginic acid, sodium alginate, or the like may be added as a disintegrant, and an anti-coagulant, a lubricant, a wetting agent, a flavoring agent, an emulsifier, an antiseptic, and the like may be additionally added.

Examples of a preparation for parenteral administration include an aqueous sterile solution, a non-aqueous solvent, a suspension solvent, an emulsion, a freeze-dried preparation, a suppository, or the like. As the non-aqueous solvent and the suspension solvent, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerol, gelatin, and the like.

The pharmaceutical composition of the present invention may be administered orally or parenterally, and, when administered parenterally, may be formulated in the form of a preparation for external application to the skin; an injection administered intraperitoneally, rectally, intravenously, muscularly, subcutaneously, or intracerebroventricularly, or via cervical intrathecal injection; a percutaneous administration agent; or a nasal inhaler according to a method known in the art.

The injection must be sterilized and protected from contamination of microorganisms such as bacteria and fungi. Examples of a suitable carrier for the injection may be, but are not limited to, a solvent or a dispersion medium including water, ethanol, polyols (for example, glycerol, propylene glycol, liquid polyethylene glycol, and the like), mixtures thereof, and/or vegetable oils. More preferably, as a suitable carrier, it is possible to use an isotonic solution such as Hank's solution, Ringer's solution, triethanolamine-containing phosphate buffered saline (PBS) or sterile water for injection, 10% ethanol, 40% propylene glycol, and 5% dextrose, and the like. To protect the injection from microbial contamination, various antimicrobial agents and antifungal agents such as a paraben, chlorobutanol, phenol, sorbic acid, and thimerosal may be additionally included. Furthermore, in most cases, the injection may additionally include an isotonic agent such as sugar or sodium chloride.

Examples of the percutaneous administration agent include a form such as an ointment, a cream, a lotion, a gel, a solution for external use, a paste, a liniment, and an aerosol. The transdermal administration as described above means that an effective amount of an active ingredient contained in a pharmaceutical composition is delivered into the skin via local administration thereof to the skin.

In the case of a preparation for inhalation, the fusion protein used according to the present invention may be conveniently delivered in the form of an aerosol spray from a pressurized pack or a nebulizer by using a suitable propellant, for example, dichlorofluoromethane, trichlorofluoromethane, dichlorotetrafluoroethane, carbon dioxide, or other suitable gases. In the case of the pressurized aerosol, a dosage unit may be determined by providing a valve for transferring a metered amount. For example, a gelatin capsule and a cartridge for use in an inhaler or insufflator may be formulated so as to contain a powder mixture of a compound and a suitable powder base such as lactose or starch. Formulations for parenteral administration are described in the document, which is a guidebook generally known in all pharmaceutical chemistry fields (Remington's Pharmaceutical Science, 15th Edition, 1975. Mack Publishing Company, Easton, Pa. 18042, Chapter 87: Blaug, Seymour).

The pharmaceutical composition of the present invention is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration routes, excretion rate, treatment periods, and simultaneously used drugs, and other factors well known in the medical field. The pharmaceutical composition of the present invention may be administered as an individual therapeutic agent or in combination with other therapeutic agents, may be administered sequentially or simultaneously with therapeutic agents in the related art, and may be administered in a single dose or multiple doses. That is, the total effective amount of the composition of the present invention may be administered to a patient in a single dose or may be administered by a fractionated treatment protocol, in which multiple doses are administered over a long period of time. It is important to administer the composition in a minimum amount that can obtain the maximum effect without any side effects, in consideration of all the aforementioned factors, and this amount may be easily determined by those skilled in the art.

A dosage of the pharmaceutical composition of the present invention varies according to body weight, age, gender, and health status of a patient, age of a patient, diet, administration time, administration method, excretion rate, and the severity of a disease. A daily dosage thereof may be administered parenterally in an amount of preferably 0.01 to 50 mg, and more preferably 0.1 mg to 30 mg per 1 kg of body weight a day based on HSA-Slit3 LRRD2, and a daily dosage thereof may be administered orally in a single dose or multiple doses in an amount of preferably 0.01 to 100 mg, and more preferably 0.01 to 10 mg per 1 kg of body weight a day based on the HSA-Slit3 LRRD2 of the present invention. However, since the effective amount may be increased or decreased depending on the administration route, the severity of obesity, gender, body weight, age, and the like, the dosage is not intended to limit the scope of the present invention in any way.

The pharmaceutical composition of the present invention may be used either alone or in combination with surgery, radiation therapy, hormone therapy, chemotherapy, and methods using a biological response modifier.

The pharmaceutical composition of the present invention may also be provided as a formulation for external application. When the pharmaceutical composition for preventing and treating bone-related diseases according to the present invention is used as a preparation for external application to the skin, the pharmaceutical composition may additionally contain auxiliary agents typically used in the dermatology field, such as any other ingredients typically used in the preparation for external application to the skin, such as a fatty substance, an organic solvent, a solubilizing agent, a thickener and a gelling agent, a softener, an antioxidant, a suspending agent, a stabilizer, a foaming agent, an odorant, a surfactant, water, an ionic emulsifier, a non-ionic emulsifier, a filler, a metal ion blocking agent, a chelating agent, a preservative, a vitamin, a blocking agent, a wetting agent, an essential oil, a dye, a pigment, a hydrophilic active agent, a lipophilic active agent, or a lipid vesicle. In addition, the ingredients may be introduced in an amount generally used in the dermatology field.

When the pharmaceutical composition for preventing and treating bone-related diseases according to the present invention is provided as a preparation for external application to the skin, the pharmaceutical composition may be in the form of a formulation such as an ointment, a patch, a gel, a cream, and an aerosol, but is not limited thereto.

The bone-related disease of the present invention refers to a disease that may occur due to an increase in bone resorption or a decrease in bone formation, for example, a decrease in bone mass while bone formation becomes less than bone resorption, and is more preferably any one or more selected from the group consisting of osteoporosis, fractures, bone loss, osteoarthritis, metastatic bone cancer, and Paget's disease, but is not limited thereto.

The present invention also provides a health functional food composition comprising albumin-bound LRRD2 of the Slit3 protein for prevention or alleviation of bone-related diseases. Since the composition of an active ingredient included in the health functional food composition of the present invention and effects thereof are the same as those for the above-described pharmaceutical composition, the description thereof will be omitted.

The health functional food composition according to the present invention can be prepared in various forms by typical methods known in the art. A general food can be prepared by adding the HSA-Slit3 LRRD2 fusion protein of the present invention to, without being limited to, a beverage (including an alcoholic beverage), fruit and a processed food thereof (for example: canned fruit, bottled food, jam, marmalade, and the like), fish, meat and processed food thereof (for example: ham, sausage, corned beef, and the like), bread and noodles (for example: thick wheat noodles, buckwheat noodles, instant noodles, spaghetti, macaroni, and the like), fruit juice, various drinks, cookies, wheat-gluten, dairy products (for example: butter, cheese, and the like), edible vegetable oils, margarine, vegetable protein, retort foods, frozen food and various seasonings (for example: soybean paste, soy sauce, sauce, and the like), and the like. In addition, a nutritional supplement can be prepared by adding the HSA-Slit3 LRRD2 fusion protein of the present invention to, without being limited to, a capsule, a tablet, a pill, and the like. Furthermore, for a health functional food, for example, the HSA-Slit3 LRRD2 fusion protein of the present invention itself is prepared in the form of, without being limited to, tea, juice, and drinks and can be taken by being processed into a liquid, granules, a capsule, and a powder so as to be able to be drunk (health beverage). Further, the HSA-Slit3 LRRD2 fusion protein of the present invention can be used and prepared in the form of a powder or a concentrated liquid so as to be used in the form of a food additive. In addition, the food functional composition of the present invention can be prepared in the form of a composition by mixing the HSA-Slit3 LRRD2 fusion protein of the present invention with an active ingredient known to have effects of preventing bone-related diseases and improving muscular function.

When the HSA-Slit3 LRRD2 fusion protein of the present invention is used as a health beverage, the health beverage composition can contain various flavoring agents or natural carbohydrates, and the like as additional ingredients, such as a typical beverage. The above-described natural carbohydrates may be monosaccharides such as glucose and fructose; disaccharides such as maltose and sucrose; polysaccharides such as dextrin and cyclodextrin; and sugar alcohols such as xylitol, sorbitol, and erythritol. As a sweetener, it is possible to use a natural sweetener such as thaumatin and stevia extract; a synthetic sweetener such as saccharin and aspartame, and the like. The proportion of the natural carbohydrate is generally about 0.01 to 0.04 g, and preferably about 0.02 to 0.03 g per 100 mL of the composition of the present invention.

Furthermore, the HSA-Slit3 LRRD2 fusion protein of the present invention may be contained as an active ingredient of a food composition for prevention or alleviation of bone-related diseases, and the amount thereof is an amount effective to achieve effects of preventing or alleviating bone-related diseases and is not particularly limited, but is preferably 0.01 to 100 wt % based on the total weight of the entire composition. The health functional food composition of the present invention can be prepared by mixing the HSA-Slit3 LRRD2 fusion protein of the present invention with other active ingredients known to have effects on bone-related diseases.

In addition to the aforementioned ingredients, the health functional food composition of the present invention may contain various nutrients, vitamins, electrolytes, flavoring agents, coloring agents, pectic acid, salts of pectic acid, alginic acid, salts of alginic acid, organic acids, protective colloidal thickeners, pH adjusting agents, stabilizers, preservatives, glycerin, alcohols, carbonate agents, and the like. In addition, the health food of the present invention may contain flesh for preparing natural fruit juice, fruit juice beverages, or vegetable beverages. These ingredients may be used either alone or in mixtures thereof. The proportion of these additives is not significantly important, but is generally selected within a range of 0.01 to 0.1 part by weight per 100 parts by weight of the composition of the present invention.

Hereinafter, the present invention will be described in more detail through Examples. These Examples are only for exemplifying the present invention, and it should be obvious to a person with ordinary skill in the art that the scope of the present invention is not to be interpreted as being limited by these Examples.

The abbreviations used in the examples and meanings thereof are as shown in the following Table 1.

TABLE 1

| | |
|---|---|
| CL | Systemic plasma clearance |
| T_{1/2} | Terminal half-life |
| Vₛₛ | Steady state volume of distribution |
| IV | Intravenous |
| PO | Per oral |
| Cₘₐₓ | Maximum plasma concentration observed |
| Tₘₐₓ | Time to Cₘₐₓ |
| AUC_{0-∞} | Total area under the plasma concentration time curve from zero |
| | to infinity |
| AUC₀₋ₜ | Area under the plasma concentration time curve from zero to the |
| | last quantifiable time point |
| MRT | Mean residence time |
| BA | Estimated bioavailability |
| BQL | Below Quantification Level |

### Example 1

Preparation of HSA-Slit3 LRRD2 Fusion Protein

Expression was performed by transforming Expi293F suspension cells with 1.6 mg/ml PC DNA3.1 vector SP cystatin S-HSA-Slit3 LRR D2-FLAG DNA. After cells were cultured to 4.5 to 5×10⁶ cells/ml in 125 ml of a 293F cell suspension and only the medium was replaced with a new medium, transfection was performed by reacting 400 μl of Expifectamine with 7.5 ml of (A Sample) at room temperature for 5 minutes, reacting 150 ug of DNA with 7.5 ml of Opti-mem (B Sample) at room temperature for 5 minutes, and then mixing A and B Samples to react A and B Samples at room temperature for 20 minutes. After 24 hours, cells were treated by mixing Enhancers 1 and 2, and then cultured for 7 days.

After cells were precipitated from the culture solution cultured for 7 days using a centrifuge at 4° C. and 800 rpm for 20 minutes, the supernatant was filtered with a 0.22 μm filter manufactured by Corning and used. As a resin, an anti-FLAG resin manufactured by Sigma was used. 1.2 ml of the resin was respectively used, and purification was performed at 1 ml/min at 4° C. A washing buffer using Tris glycine (TBS, pH 7.4) was flowed in an amount which is 20-fold higher than that of the resin. For elution, 200 μl of a FLAG peptide manufactured by Sigma-Aldrich and 9.8 ml of TBS were mixed and used, 8 pieces of 500 μl per fraction were obtained, protein fractions were collected, concentrated by changing the buffer to DPBS, and then the concentration was measured.

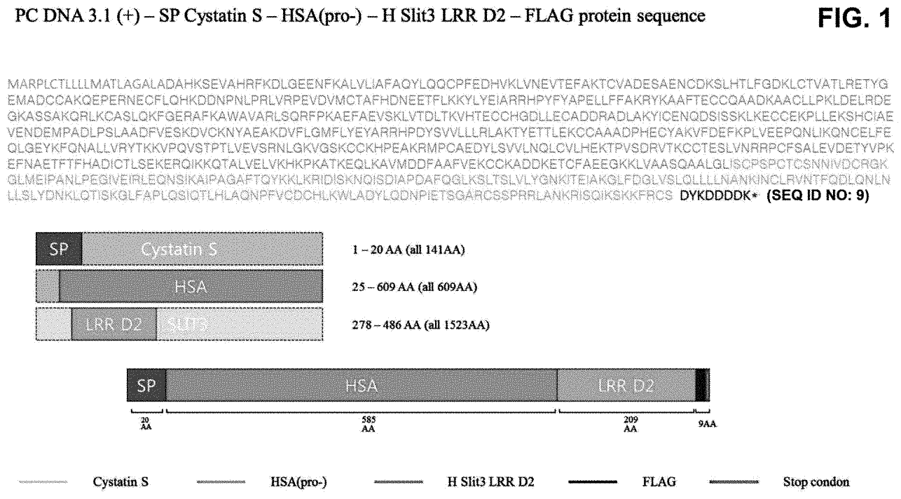

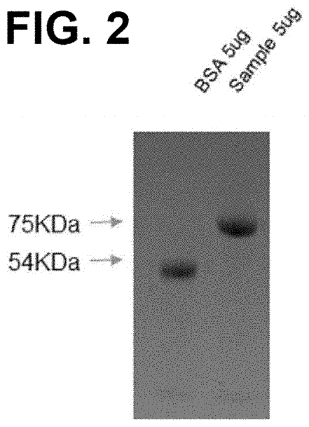

FIG. 2 illustrates the results of performing SDS-PAGE after isolating and purifying the fusion protein by the above process, confirming that the size of the fusion protein illustrated in FIG. 1, which was prepared in the present example, was 75 KDa.

### Example 2

Confirmation of Receptor Binding Ability of Various Forms of HSA-Slit3 LRRD2 Fusion Proteins

2-1. Preparation of Various Forms of HSA-Slit3 LRRD2 Fusion Proteins

Based on the preparation method of Example 1, 12 types of various HSA-Slit3 LRRD2 fusion proteins were prepared as shown in the following Table 2. As a linker, (GGGGS)₃ (SEQ ID NO: 6) was used.

TABLE 2

| | | Presence | | |
|---|---|---|---|---|
| | | or | | |
| Type of | Terminus | absence | | |
| fusion | bound to | of | Type of | |
| protein | HSA | linker | LRRD2 | Final form |
| LRRD2-1 | N terminus | None | Fragment | HSA-Fragment |
| | | | (68 a.a.) | LRRD2 |
| LRRD2-2 | N terminus | None | Intermediate | HSA-Intermediate |
| | | | (130 a.a) | LRRD2 |
| LRRD2-3 | N terminus | None | Full-length | HSA-Full-length |
| | | | (209 a.a) | LRRD2 |
| LRRD2-4 | N terminus | Present | Fragment | HSA-Linker- |
| | | | (68 a.a.) | Fragment LRRD2 |
| LRRD2-5 | N terminus | Present | Intermediate | HSA-Linker- |
| | | | (130 a.a) | Intermediate LRRD2 |
| LRRD2-6 | N terminus | Present | Full-length | HSA-Linker-Full- |
| | | | (209 a.a) | length LRRD2 |
| LRRD2-7 | C terminus | None | Fragment | Fragment |
| | | | (68 a.a.) | LRRD2-HSA |
| LRRD2-8 | C terminus | None | Intermediate | Intermediate |
| | | | (130 a.a) | LRRD2-HSA |
| LRRD2-9 | C terminus | None | Full-length | Full-length |
| | | | (209 a.a) | LRRD2-HSA |
| LRRD2-10 | C terminus | Present | Fragment | Fragment LRRD2- |
| | | | (68 a.a.) | Linker-HSA |
| LRRD2-11 | C terminus | Present | Intermediate | Intermediate |
| | | | (130 a.a) | LRRD2-Linker-HSA |
| LRRD2-12 | C terminus | Present | Full-length | Full-length |
| | | | (209 a.a) | LRRD2-Linker-HSA |

The amino acid sequences of the 12 types of HSA-Slit3LRRD2 fusion proteins are shown in Table 3.

TABLE 3

| Type of | | |
|---|---|---|
| fusion | Amino acid | SEQ ID |
| protein | sequence | NO |
| LRRD2-1 | MMARPLCTLLLLMATLAGAL | 7 |
| | ADAHKSEVAHRFKDLGEENF | |
| | KALVLIAFAQYLQQCPFEDH | |
| | VKLVNEVTEFAKTCVADESA | |
| | ENCDKSLHTLFGDKLCTVAT | |
| | LRETYGEMADCCAKQEPERN | |
| | ECFLQHKDDNPNLPRLVRPE | |
| | VDVMCTAFHDNEETFLKKYL | |
| | YEIARRHPYFYAPELLFFAK | |
| | RYKAAFTECCQAADKAACLL | |
| | PKLDELRDEGKASSAKQRLK | |
| | CASLQKFGERAFKAWAVARL | |
| | SQRFPKAEFAEVSKLVTDLT | |
| | KVHTECCHGDLLECADDRAD | |
| | LAKYICENQDSISSKLKECC | |
| | EKPLLEKSHCIAEVENDEMP | |
| | ADLPSLAADFVESKDVCKNY | |
| | AEAKDVFLGMFLYEYARRHP | |
| | DYSVVLLLRLAKTYETTLEK | |
| | CCAAADPHECYAKVFDEFKP | |
| | LVEEPQNLIKQNCELFEQLG | |
| | EYKFQNALLVRYTKKVPQVS | |
| | TPTLVEVSRNLGKVGSKCCK | |
| | HPEAKRMPCAEDYLSVVLNQ | |
| | LCVLHEKTPVSDRVTKCCTE | |
| | SLVNRRPCFSALEVDETYVP | |
| | KEFNAETFTFHADICTLSEK | |
| | ERQIKKQTALVELVKHKPKA | |
| | TKEQLKAVMDDFAAFVEKCC | |
| | KADDKETCFAEEGKKLVAAS | |
| | QAALGLLTSLVLYGNKITEI | |
| | AKGLFDGLVSLQLLLLNANK | |
| | INCLRVNTFQDLQNLNLLSL | |
| | YDNKLQTISKGLFADYKDDD | |
| | DK | |
| LRRD2-2 | MARPLCTLLLLMATLAGALA | 8 |
| | DAHKSEVAHRFKDLGEENFK | |
| | ALVLIAFAQYLQQCPFEDHV | |
| | KLVNEVTEFAKTCVADESAE | |
| | NCDKSLHTLFGDKLCTVATL | |
| | RETYGEMADCCAKQEPERNE | |
| | CFLQHKDDNPNLPRLVRPEV | |
| | DVMCTAFHDNEETFLKKYLY | |
| | EIARRHPYFYAPELLFFAKR | |
| | YKAAFTECCQAADKAACLLP | |
| | KLDELRDEGKASSAKQRLKC | |
| | ASLQKFGERAFKAWAVARLS | |
| | QRFPKAEFAEVSKLVTDLTK | |
| | VHTECCHGDLLECADDRADL | |
| | AKYICENQDSISSKLKECCE | |
| | KPLLEKSHCIAEVENDEMPA | |
| | DLPSLAADFVESKDVCKNYA | |
| | EAKDVFLGMFLYEYARRHPD | |
| | YSVVLLLRLAKTYETTLEKC | |
| | CAAADPHECYAKVFDEFKPL | |
| | VEEPQNLIKQNCELFEQLGE | |
| | YKFQNALLVRYTKKVPQVST | |
| | PTLVEVSRNLGKVGSKCCKH | |
| | PEAKRMPCAEDYLSVVLNQL | |
| | CVLHEKTPVSDRVTKCCTES | |
| | LVNRRPCFSALEVDETYVPK | |
| | EFNAETFTFHADICTLSEKE | |
| | RQIKKQTALVELVKHKPKAT | |
| | KEQLKAVMDDFAAFVEKCCK | |
| | ADDKETCFAEEGKKLVAASQ | |
| | AALGLIVEIRLEQNSIKAIP | |
| | AGAFTQYKKLKRIDISKNQI | |
| | SDIAPDAFQGLKSLTSLVLY | |
| | GNKITEIAKGLFDGLVSLQL | |
| | LLLNANKINCLRVNTFQDLQ | |
| | NLNLLSLYDNKLQTISKGLF | |
| | APLQSIQTLHLAQNPDYKDD | |
| | DDK | |
| LRRD2-3 | MARPLCTLLLLMATLAGALA | 9 |
| | DAHKSEVAHRFKDLGEENFK | |
| | ALVLIAFAQYLQQCPFEDHV | |
| | KLVNEVTEFAKTCVADESAE | |
| | NCDKSLHTLFGDKLCTVATL | |
| | RETYGEMADCCAKQEPERNE | |
| | CFLQHKDDNPNLPRLVRPEV | |
| | DVMCTAFHDNEETFLKKYLY | |
| | EIARRHPYFYAPELLFFAKR | |
| | YKAAFTECCQAADKAACLLP | |
| | KLDELRDEGKASSAKQRLKC | |
| | ASLQKFGERAFKAWAVARLS | |
| | QRFPKAEFAEVSKLVTDLTK | |
| | VHTECCHGDLLECADDRADL | |
| | AKYICENQDSISSKLKECCE | |
| | KPLLEKSHCIAEVENDEMPA | |
| | DLPSLAADFVESKDVCKNYA | |
| | EAKDVFLGMFLYEYARRHPD | |
| | YSVVLLLRLAKTYETTLEKC | |
| | CAAADPHECYAKVFDEFKPL | |
| | VEEPQNLIKQNCELFEQLGE | |
| | YKFQNALLVRYTKKVPQVST | |
| | PTLVEVSRNLGKVGSKCCKH | |
| | PEAKRMPCAEDYLSVVLNQL | |
| | CVLHEKTPVSDRVTKCCTES | |
| | LVNRRPCFSALEVDETYVPK | |
| | EFNAETFTFHADICTLSEKE | |
| | RQIKKQTALVELVKHKPKAT | |
| | KEQLKAVMDDFAAFVEKCCK | |
| | ADDKETCFAEEGKKLVAASQ | |
| | AALGLISCPSPCTCSNNIVD | |
| | CRGKGLMEIPANLPEGIVEI | |
| | RLEQNSIKAIPAGAFTQYKK | |
| | LKRIDISKNQISDIAPDAFQ | |
| | GLKSLTSLVLYGNKITEIAK | |
| | GLFDGLVSLQLLLLNANKIN | |
| | CLRVNTFQDLQNLNLLSLYD | |
| | NKLQTISKGLFAPLQSIQTL | |
| | HLAQNPFVCDCHLKWLADYL | |
| | QDNPIETSGARCSSPRRLAN | |
| | KRISQIKSKKFRCSDYKDDD | |
| | DK | |
| LRRD2-4 | MARPLCTLLLLMATLAGALA | 10 |
| | DAHKSEVAHRFKDLGEENFK | |
| | ALVLIAFAQYLQQCPFEDHV | |
| | KLVNEVTEFAKTCVADESAE | |
| | NCDKSLHTLFGDKLCTVATL | |
| | RETYGEMADCCAKQEPERNE | |
| | CFLQHKDDNPNLPRLVRPEV | |
| | DVMCTAFHDNEETFLKKYLY | |
| | EIARRHPYFYAPELLFFAKR | |
| | YKAAFTECCQAADKAACLLP | |
| | KLDELRDEGKASSAKQRLKC | |
| | ASLQKFGERAFKAWAVARLS | |
| | QRFPKAEFAEVSKLVTDLTK | |
| | VHTECCHGDLLECADDRADL | |
| | AKYICENQDSISSKLKECCE | |
| | KPLLEKSHCIAEVENDEMPA | |
| | DLPSLAADFVESKDVCKNYA | |
| | EAKDVFLGMFLYEYARRHPD | |
| | YSVVLLLRLAKTYETTLEKC | |
| | CAAADPHECYAKVFDEFKPL | |
| | VEEPQNLIKQNCELFEQLGE | |
| | YKFQNALLVRYTKKVPQVST | |
| | PTLVEVSRNLGKVGSKCCKH | |
| | PEAKRMPCAEDYLSVVLNQL | |
| | CVLHEKTPVSDRVTKCCTES | |
| | LVNRRPCFSALEVDETYVPK | |
| | EFNAETFTFHADICTLSEKE | |
| | RQIKKQTALVELVKHKPKAT | |
| | KEQLKAVMDDFAAFVEKCCK | |
| | ADDKETCFAEEGKKLVAASQ | |
| | AALGLGGGGSGGGGSGGGGS | |
| | LTSLVLYGNKITEIAKGLFD | |
| | GLVSLQLLLLNANKINCLRV | |
| | NTFQDLQNLNLLSLYDNKLQ | |
| | TISKGLFADYKDDDDK | |
| LRRD2-5 | MARPLCTLLLLMATLAGALA | 11 |
| | DAHKSEVAHRFKDLGEENFK | |
| | ALVLIAFAQYLQQCPFEDHV | |
| | KLVNEVTEFAKTCVADESAE | |
| | NCDKSLHTLFGDKLCTVATL | |
| | RETYGEMADCCAKQEPERNE | |
| | CFLQHKDDNPNLPRLVRPEV | |
| | DVMCTAFHDNEETFLKKYLY | |
| | EIARRHPYFYAPELLFFAKR | |
| | YKAAFTECCQAADKAACLLP | |
| | KLDELRDEGKASSAKQRLKC | |
| | ASLQKFGERAFKAWAVARLS | |
| | QRFPKAEFAEVSKLVTDLTK | |
| | VHTECCHGDLLECADDRADL | |
| | AKYICENQDSISSKLKECCE | |
| | KPLLEKSHCIAEVENDEMPA | |
| | DLPSLAADFVESKDVCKNYA | |
| | EAKDVFLGMFLYEYARRHPD | |
| | YSVVLLLRLAKTYETTLEKC | |
| | CAAADPHECYAKVFDEFKPL | |
| | VEEPQNLIKQNCELFEQLGE | |
| | YKFQNALLVRYTKKVPQVST | |
| | PTLVEVSRNLGKVGSKCCKH | |
| | PEAKRMPCAEDYLSVVLNQL | |
| | CVLHEKTPVSDRVTKCCTES | |
| | LVNRRPCFSALEVDETYVPK | |
| | EFNAETFTFHADICTLSEKE | |
| | RQIKKQTALVELVKHKPKAT | |
| | KEQLKAVMDDFAAFVEKCCK | |
| | ADDKETCFAEEGKKLVAASQ | |
| | AALGLGGGGSGGGGSGGGGS | |
| | IVEIRLEQNSIKAIPAGAFT | |
| | QYKKLKRIDISKNQISDIAP | |
| | DAFQGLKSLTSLVLYGNKIT | |
| | EIAKGLFDGLVSLQLLLLNA | |
| | NKINCLRVNTFQDLQNLNLL | |
| | SLYDNKLQTISKGLFAPLQS | |
| | IQTLHLAQNPDYKDDDDK | |
| LRRD2-6 | MARPLCTLLLLMATLAGALA | 12 |
| | DAHKSEVAHRFKDLGEENFK | |
| | ALVLIAFAQYLQQCPFEDHV | |
| | KLVNEVTEFAKTCVADESAE | |
| | NCDKSLHTLFGDKLCTVATL | |
| | RETYGEMADCCAKQEPERNE | |
| | CFLQHKDDNPNLPRLVRPEV | |
| | DVMCTAFHDNEETFLKKYLY | |
| | EIARRHPYFYAPELLFFAKR | |
| | YKAAFTECCQAADKAACLLP | |
| | KLDELRDEGKASSAKQRLKC | |
| | ASLQKFGERAFKAWAVARLS | |
| | QRFPKAEFAEVSKLVTDLTK | |
| | VHTECCHGDLLECADDRADL | |
| | AKYICENQDSISSKLKECCE | |
| | KPLLEKSHCIAEVENDEMPA | |
| | DLPSLAADFVESKDVCKNYA | |
| | EAKDVFLGMFLYEYARRHPD | |
| | YSVVLLLRLAKTYETTLEKC | |
| | CAAADPHECYAKVFDEFKPL | |
| | VEEPQNLIKQNCELFEQLGE | |
| | YKFQNALLVRYTKKVPQVST | |
| | PTLVEVSRNLGKVGSKCCKH | |
| | PEAKRMPCAEDYLSVVLNQL | |
| | CVLHEKTPVSDRVTKCCTES | |
| | LVNRRPCFSALEVDETYVPK | |
| | EFNAETFTFHADICTLSEKE | |
| | RQIKKQTALVELVKHKPKAT | |
| | KEQLKAVMDDFAAFVEKCCK | |
| | ADDKETCFAEEGKKLVAASQ | |
| | AALGLGGGGSGGGGSGGGGS | |
| | ISCPSPCTCSNNIVDCRGKG | |
| | LMEIPANLPEGIVEIRLEQN | |
| | SIKAIPAGAFTQYKKLKRID | |
| | ISKNQISDIAPDAFQGLKSL | |
| | TSLVLYGNKITEIAKGLFDG | |
| | LVSLQLLLLNANKINCLRVN | |
| | TFQDLQNLNLLSLYDNKLQT | |
| | ISKGLFAPLQSIQTLHLAQN | |
| | PFVCDCHLKWLADYLQDNPI | |
| | ETSGARCSSPRRLANKRISQ | |
| | IKSKKFRCSDYKDDDDK | |
| LRRD2-7 | MARPLCTLLLLMATLAGALA | 13 |
| | LTSLVLYGNKITEIAKGLFD | |
| | GLVSLQLLLLNANKINCLRV | |
| | NTFQDLQNLNLLSLYDNKLQ | |
| | TISKGLFADAHKSEVAHRFK | |
| | DLGEENFKALVLIAFAQYLQ | |
| | QCPFEDHVKLVNEVTEFAKT | |
| | CVADESAENCDKSLHTLFGD | |
| | KLCTVATLRETYGEMADCCA | |
| | KQEPERNECFLQHKDDNPNL | |
| | PRLVRPEVDVMCTAFHDNEE | |
| | TFLKKYLYEIARRHPYFYAP | |
| | ELLFFAKRYKAAFTECCQAA | |
| | DKAACLLPKLDELRDEGKAS | |
| | SAKQRLKCASLQKFGERAFK | |
| | AWAVARLSQRFPKAEFAEVS | |
| | KLVTDLTKVHTECCHGDLLE | |
| | CADDRADLAKYICENQDSIS | |
| | SKLKECCEKPLLEKSHCIAE | |
| | VENDEMPADLPSLAADFVES | |
| | KDVCKNYAEAKDVFLGMFLY | |
| | EYARRHPDYSVVLLLRLAKT | |
| | YETTLEKCCAAADPHECYAK | |
| | VFDEFKPLVEEPQNLIKQNC | |
| | ELFEQLGEYKFQNALLVRYT | |
| | KKVPQVSTPTLVEVSRNLGK | |
| | VGSKCCKHPEAKRMPCAEDY | |
| | LSVVLNQLCVLHEKTPVSDR | |
| | VTKCCTESLVNRRPCFSALE | |
| | VDETYVPKEFNAETFTFHAD | |
| | ICTLSEKERQIKKQTALVEL | |
| | VKHKPKATKEQLKAVMDDFA | |
| | AFVEKCCKADDKETCFAEEG | |
| | KKLVAASQAALGLGGGGSGG | |
| | GGSGGGGSDYKDDDDK | |
| LRRD2-8 | MARPLCTLLLLMATLAGALA | 14 |
| | IVEIRLEQNSIKAIPAGAFT | |
| | QYKKLKRIDISKNQISDIAP | |
| | DAFQGLKSLTSLVLYGNKIT | |
| | EIAKGLFDGLVSLQLLLLNA | |
| | NKINCLRVNTFQDLQNLNLL | |
| | SLYDNKLQTISKGLFAPLQS | |
| | IQTLHLAQNPDAHKSEVAHR | |
| | FKDLGEENFKALVLIAFAQY | |
| | LQQCPFEDHVKLVNEVTEFA | |
| | KTCVADESAENCDKSLHTLF | |
| | GDKLCTVATLRETYGEMADC | |
| | CAKQEPERNECFLQHKDDNP | |
| | NLPRLVRPEVDVMCTAFHDN | |
| | EETFLKKYLYEIARRHPYFY | |
| | APELLFFAKRYKAAFTECCQ | |
| | AADKAACLLPKLDELRDEGK | |
| | ASSAKQRLKCASLQKFGERA | |
| | FKAWAVARLSQRFPKAEFAE | |
| | VSKLVTDLTKVHTECCHGDL | |
| | LECADDRADLAKYICENQDS | |
| | ISSKLKECCEKPLLEKSHCI | |
| | AEVENDEMPADLPSLAADFV | |
| | ESKDVCKNYAEAKDVFLGMF | |
| | LYEYARRHPDYSVVLLLRLA | |
| | KTYETTLEKCCAAADPHECY | |
| | AKVFDEFKPLVEEPQNLIKQ | |
| | NCELFEQLGEYKFQNALLVR | |
| | YTKKVPQVSTPTLVEVSRNL | |
| | GKVGSKCCKHPEAKRMPCAE | |
| | DYLSVVLNQLCVLHEKTPVS | |
| | DRVTKCCTESLVNRRPCFSA | |
| | LEVDETYVPKEFNAETFTFH | |
| | ADICTLSEKERQIKKQTALV | |
| | ELVKHKPKATKEQLKAVMDD | |
| | FAAFVEKCCKADDKETCFAE | |
| | EGKKLVAASQAALGLGGGGS | |
| | GGGGSGGGGSDYKDDDDK | |
| LRRD2-9 | MARPLCTLLLLMATLAGALA | 15 |
| | ISCPSPCTCSNNIVDCRGKG | |
| | LMEIPANLPEGIVEIRLEQN | |
| | SIKAIPAGAFTQYKKLKRID | |
| | ISKNQISDIAPDAFQGLKSL | |
| | TSLVLYGNKITEIAKGLFDG | |
| | LVSLQLLLLNANKINCLRVN | |
| | TFQDLQNLNLLSLYDNKLQT | |
| | ISKGLFAPLQSIQTLHLAQN | |
| | PFVCDCHLKWLADYLQDNPI | |
| | ETSGARCSSPRRLANKRISQ | |
| | IKSKKFRCSDAHKSEVAHRF | |
| | KDLGEENFKALVLIAFAQYL | |
| | QQCPFEDHVKLVNEVTEFAK | |
| | TCVADESAENCDKSLHTLFG | |
| | DKLCTVATLRETYGEMADCC | |
| | AKQEPERNECFLQHKDDNPN | |
| | LPRLVRPEVDVMCTAFHDNE | |
| | ETFLKKYLYEIARRHPYFYA | |
| | PELLFFAKRYKAAFTECCQA | |
| | ADKAACLLPKLDELRDEGKA | |
| | SSAKQRLKCASLQKFGERAF | |
| | KAWAVARLSQRFPKAEFAEV | |
| | SKLVTDLTKVHTECCHGDLL | |
| | ECADDRADLAKYICENQDSI | |
| | SSKLKECCEKPLLEKSHCIA | |
| | EVENDEMPADLPSLAADFVE | |
| | SKDVCKNYAEAKDVFLGMFL | |
| | YEYARRHPDYSVVLLLRLAK | |
| | TYETTLEKCCAAADPHECYA | |
| | KVFDEFKPLVEEPQNLIKQN | |
| | CELFEQLGEYKFQNALLVRY | |
| | TKKVPQVSTPTLVEVSRNLG | |
| | KVGSKCCKHPEAKRMPCAED | |
| | YLSVVLNQLCVLHEKTPVSD | |
| | RVTKCCTESLVNRRPCFSAL | |
| | EVDETYVPKEFNAETFTFHA | |
| | DICTLSEKERQIKKQTALVE | |
| | LVKHKPKATKEQLKAVMDDF | |
| | AAFVEKCCKADDKETCFAEE | |
| | GKKLVAASQAALGLGGGGSG | |
| | GGGSGGGGSDYKDDDDK | |
| LRRD2-10 | MARPLCTLLLLMATLAGALA | 16 |
| | LTSLVLYGNKITEIAKGLFD | |
| | GLVSLQLLLLNANKINCLRV | |
| | NTFQDLQNLNLLSLYDNKLQ | |
| | TISKGLFAGGGGSGGGGSGG | |
| | GGSDAHKSEVAHRFKDLGEE | |
| | NFKALVLIAFAQYLQQCPFE | |
| | DHVKLVNEVTEFAKTCVADE | |
| | SAENCDKSLHTLFGDKLCTV | |
| | ATLRETYGEMADCCAKQEPE | |
| | RNECFLQHKDDNPNLPRLVR | |
| | PEVDVMCTAFHDNEETFLKK | |
| | YLYEIARRHPYFYAPELLFF | |
| | AKRYKAAFTECCQAADKAAC | |
| | LLPKLDELRDEGKASSAKQR | |
| | LKCASLQKFGERAFKAWAVA | |
| | RLSQRFPKAEFAEVSKLVTD | |
| | LTKVHTECCHGDLLECADDR | |
| | ADLAKYICENQDSISSKLKE | |
| | CCEKPLLEKSHCIAEVENDE | |
| | MPADLPSLAADFVESKDVCK | |
| | NYAEAKDVFLGMFLYEYARR | |
| | HPDYSVVLLLRLAKTYETTL | |
| | EKCCAAADPHECYAKVFDEF | |
| | KPLVEEPQNLIKQNCELFEQ | |
| | LGEYKFQNALLVRYTKKVPQ | |
| | VSTPTLVEVSRNLGKVGSKC | |
| | CKHPEAKRMPCAEDYLSVVL | |
| | NQLCVLHEKTPVSDRVTKCC | |
| | TESLVNRRPCFSALEVDETY | |
| | VPKEFNAETFTFHADICTLS | |
| | EKERQIKKQTALVELVKHKP | |
| | KATKEQLKAVMDDFAAFVEK | |
| | CCKADDKETCFAEEGKKLVA | |
| | ASQAALGLGGGGSGGGGSGG | |
| | GGSDYKDDDDK | |
| LRRD2-11 | MARPLCTLLLLMATLAGALA | 17 |
| | IVEIRLEQNSIKAIPAGAFT | |
| | QYKKLKRIDISKNQISDIAP | |
| | DAFQGLKSLTSLVLYGNKIT | |
| | EIAKGLFDGLVSLQLLLLNA | |
| | NKINCLRVNTFQDLQNLNLL | |
| | SLYDNKLQTISKGLFAPLQS | |
| | IQTLHLAQNPGGGGSGGGGS | |
| | GGGGSDAHKSEVAHRFKDLG | |
| | EENFKALVLIAFAQYLQQCP | |
| | FEDHVKLVNEVTEFAKTCVA | |
| | DESAENCDKSLHTLFGDKLC | |
| | TVATLRETYGEMADCCAKQE | |
| | PERNECFLQHKDDNPNLPRL | |
| | VRPEVDVMCTAFHDNEETFL | |
| | KKYLYEIARRHPYFYAPELL | |
| | FFAKRYKAAFTECCQAADKA | |
| | ACLLPKLDELRDEGKASSAK | |
| | QRLKCASLQKFGERAFKAWA | |
| | VARLSQRFPKAEFAEVSKLV | |
| | TDLTKVHTECCHGDLLECAD | |
| | DRADLAKYICENQDSISSKL | |
| | KECCEKPLLEKSHCIAEVEN | |
| | DEMPADLPSLAADFVESKDV | |
| | CKNYAEAKDVFLGMFLYEYA | |
| | RRHPDYSVVLLLRLAKTYET | |
| | TLEKCCAAADPHECYAKVFD | |
| | EFKPLVEEPQNLIKQNCELF | |
| | EQLGEYKFQNALLVRYTKKV | |
| | PQVSTPTLVEVSRNLGKVGS | |
| | KCCKHPEAKRMPCAEDYLSV | |
| | VLNQLCVLHEKTPVSDRVTK | |
| | CCTESLVNRRPCFSALEVDE | |
| | TYVPKEFNAETFTFHADICT | |
| | LSEKERQIKKQTALVELVKH | |
| | KPKATKEQLKAVMDDFAAFV | |
| | EKCCKADDKETCFAEEGKKL | |
| | VAASQAALGLGGGGSGGGGS | |
| | GGGGSDYKDDDDK | |
| LRRD2-12 | MARPLCTLLLLMATLAGALA | 18 |
| | ISCPSPCTCSNNIVDCRGKG | |
| | LMEIPANLPEGIVEIRLEQN | |
| | SIKAIPAGAFTQYKKLKRID | |
| | ISKNQISDIAPDAFQGLKSL | |
| | TSLVLYGNKITEIAKGLFDG | |
| | LVSLQLLLLNANKINCLRVN | |
| | TFQDLQNLNLLSLYDNKLQT | |
| | ISKGLFAPLQSIQTLHLAQN | |
| | PFVCDCHLKWLADYLQDNPI | |
| | ETSGARCSSPRRLANKRISQ | |
| | IKSKKFRCSGGGGSGGGGSG | |
| | GGGSDAHKSEVAHRFKDLGE | |
| | ENFKALVLIAFAQYLQQCPF | |
| | EDHVKLVNEVTEFAKTCVAD | |
| | ESAENCDKSLHTLFGDKLCT | |
| | VATLRETYGEMADCCAKQEP | |
| | ERNECFLQHKDDNPNLPRLV | |
| | RPEVDVMCTAFHDNEETFLK | |
| | KYLYEIARRHPYFYAPELLF | |
| | FAKRYKAAFTECCQAADKAA | |
| | CLLPKLDELRDEGKASSAKQ | |
| | RLKCASLQKFGERAFKAWAV | |
| | ARLSQRFPKAEFAEVSKLVT | |
| | DLTKVHTECCHGDLLECADD | |
| | RADLAKYICENQDSISSKLK | |
| | ECCEKPLLEKSHCIAEVEND | |
| | EMPADLPSLAADFVESKDVC | |
| | KNYAEAKDVFLGMFLYEYAR | |
| | RHPDYSVVLLLRLAKTYETT | |
| | LEKCCAAADPHECYAKVFDE | |
| | FKPLVEEPQNLIKQNCELFE | |
| | QLGEYKFQNALLVRYTKKVP | |
| | QVSTPTLVEVSRNLGKVGSK | |
| | CCKHPEAKRMPCAEDYLSVV | |
| | LNQLCVLHEKTPVSDRVTKC | |
| | CTESLVNRRPCFSALEVDET | |
| | YVPKEFNAETFTFHADICTL | |
| | SEKERQIKKQTALVELVKHK | |
| | PKATKEQLKAVMDDFAAFVE | |
| | KCCKADDKETCFAEEGKKLV | |
| | AASQAALGLGGGGSGGGGSG | |
| | GGGSDYKDDDDK | |

The underlined sequence in Table 3 is a GS linker linking HSA and LRRD2, and the bold sequence is a GS linker linking a sequence added to the C-terminus in order to express the fusion protein in its final form.

2-2. Confirmation of Receptor Binding Ability of Various Forms of HSA-Slit3 LRRD2 Fusion Proteins

The action of Slit3 LRRD2 on bone cells is mediated through Robo1 and Robo2 receptors. Therefore, in the present example, the Robo1 receptor binding ability of the 12 types of HSA-Slit3 LRRD2 fusion proteins prepared in Example 2-1 was confirmed. The binding ability of the 12 types of HSA-Slit3 LRRD2 fusion proteins to the receptor was quantified using an ELISA system. Detailed conditions are as follows.

96-well Maxisorp microtiter plates (manufactured by NUNC) were coated with the 12 types of HSA-Slit3 LRRD2 fusion proteins at 4° C. for 18 hours at 0, 1, 10, 100, and 1000 nM per well, in consideration of the molecular weight. The coated material was washed three times using PBS containing 0.05% Tween 20 (PBST). Blocking was performed with PBST supplemented with 1% BSA at room temperature for 2 hours to block non-specific binding. The coated material was washed three times with PBST to remove a blocking buffer. After washing, 30 ug of a protein obtained from an osteoblastic cell line, MC3T3-E1, was allowed to adhere (lysis buffer: 0.5% NP40, 50 mM Tris pH 7.5, 150 mM NaCl, 1 mM EDTA, 0.2 mM NaF, 1 mM Na₃VO₄, 1 mM DTT, 1 mM PMSF, and a proteinase inhibitor cocktail) at room temperature for 2 hours. After washing three times with PBST, a Robo1 antibody (abcam: ab7279) diluted with 0.1% BSA at 1:1000 was adhered thereto at room temperature for 2 hours. After washing three times with PBST, an HRP-binding antibody (cell signaling: 7074) diluted with 0.1% BSA at 1:2000 was adhered thereto at room temperature for 2 hours. After washing five times with PBST, a reaction was performed with a TMB solution at 37° C. for 30 minutes. To stop the reaction, 100 μl of 1 N H₂SO₄ was used, and absorbance was measured at 450 nm.

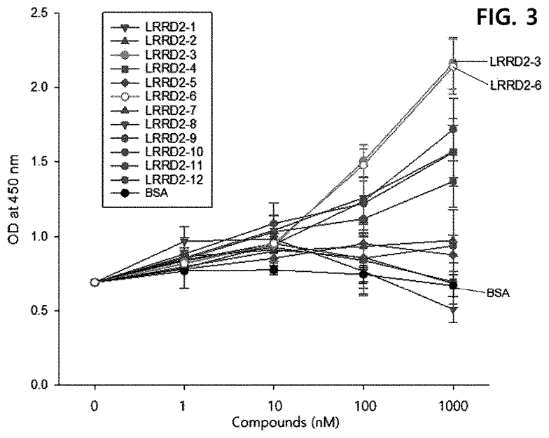

As a result, as illustrated in FIG. 3, it was confirmed that the receptor binding ability of LRRD2-3 and LRRD2-6 was the best.

### Example 3

Confirmation of Cytological Efficacy of Various Forms of HSA-Slit3 LRRD2 Fusion Proteins

In the present example, the cytological efficacy of the 12 types of HSA-Slit3LRRD2 fusion proteins prepared in Example 2-1 was confirmed by observing the osteoblast migration ability and the osteoclast differentiation ability according to the treatment of the 12 types of HSA-Slit3 LRRD2 fusion proteins.

3-1. Measurement of Osteoblast Migration Ability

A Boyden chamber system (Transwell, 8 um pores) was used to measure the cell migration ability. After an osteoblastic cell line MC3T3-E1 (1×10⁵) was diluted in an MEM alpha medium supplemented with 0.2% FBS and attached to an inner chamber for 6 hours, an outer chamber was treated with a drug for 24 hours. After invaded cells were treated with a crystal violet solution containing a fixing solution for 10 minutes, the number of cells was measured under an optical microscope.

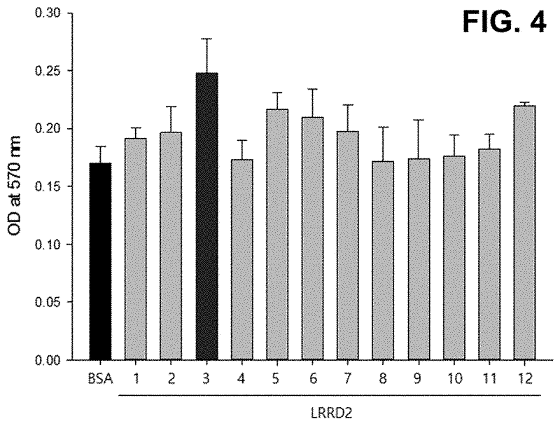

As a result, as illustrated in FIG. 4, it was confirmed that the osteoblast migration ability of LRRD2-3 was the best.

3-2. Measurement of Differentiation Ability of Osteoclasts

Osteoclast progenitor cells were extracted from the femurs and tibias of 6-week-old ICR mice, and then cultured in an incubator at 37° C. for 18 hours. Only floating cells were collected and treated with 30 ng/ml M-CSF and 30 ng/ml RANKL to differentiate into osteoclasts. After 4 days, the cells were reacted with a TRAP staining solution (leukocyte acid phosphatase) for 10 minutes, and then the number of cells was measured by considering multinucleated cells having 3 or more nuclei stained with TRAP as osteoclasts under an optical microscope.

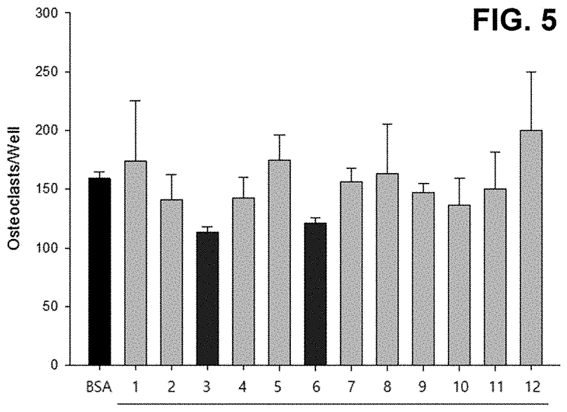

As a result, as illustrated in FIG. 5, it was confirmed that the osteoclast differentiation inhibiting ability of LRRD2-3 and LRRD2-6 was better than the remaining 10 types of HSA-Slit3 LRRD2 fusion proteins.

### Example 4

Confirmation of Cytological Efficacy of HSA-Slit3 LRRD2 Fusion Protein

In the present example, based on the results of Examples 2 and 3, LRRD2-3, which has the best efficacy in cells, was selected, and observed by comparing its osteoblast migration ability, b-catenin activity of osteoblasts, and osteoclast differentiation inhibiting ability with those of albumin-unbound Slit3 LRRD2.

4-1. Measurement of Osteoblast Migration Ability

A Boyden chamber system (Transwell, 8 um pores) was used to measure the migration ability of cells. After an osteoblastic cell line MC3T3-ET (1×10⁵) was diluted in an MEM alpha medium supplemented with 0.2% FBS and attached to an inner chamber for 6 hours, an outer chamber was treated with a drug for 24 hours. After invaded cells were treated with a crystal violet solution containing a fixing solution for 10 minutes, the number of cells was measured under an optical microscope.

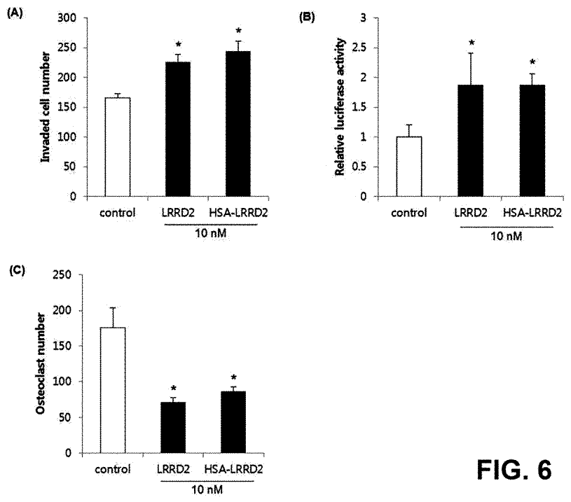

As a result, as illustrated in FIG. 6A, LRRD2-3 promoted osteoblast migration ability to the same extent as albumin-unbound Slit3 LRRD2.

4-2. Measurement of b-catenin activity of osteoblasts After an MC3T3-ET cell line (2×10⁴ cells/well) was cultured in a 24-well plate for 18 hours, 100 ng of 8× SuperTOPFlash and 10 ng of a Renilla reporter plasmid were transfected into the cells. After 48 hours, luciferase activity was measured using a dual luciferase reporter assay kit.

As a result, as illustrated in FIG. 6B, LRRD2-3 Activated b-Catenin of osteoblasts to the same extent as albumin-unbound Slit3 LRRD2.

4-3. Measurement of Differentiation Ability of Osteoclasts

Osteoclast progenitor cells were extracted from the femurs and tibias of 6-week-old ICR mice, and then cultured in an incubator at 37° C. for 18 hours. Only floating cells were collected and treated with 30 ng/ml M-CSF and 30 ng/ml RANKL to differentiate into osteoclasts. After 4 days, the cells were reacted with a TRAP staining solution (leukocyte acid phosphatase) for 10 minutes, and then the number of cells was measured by considering multinucleated cells having 3 or more nuclei stained with TRAP as osteoclasts under an optical microscope.

As a result, as illustrated in FIG. 6C, LRRD2-3 suppressed osteoclast differentiation to the same extent as albumin-unbound Slit3 LRRD2.

### Example 5

Pharmacokinetic Studies of Slit3 LRRD2 and HSA-Slit3 LRRD2 Fusion Proteins in Mice

A pharmacokinetic study is a part of new drug development processes, and aims to obtain information on the absorption, distribution, metabolism and excretion of a test drug by assessing changes in drug concentration in the body over time. In the present example, pharmacokinetic properties were confirmed in mice after a single intravenous administration of Slit3 LRRD2-3 and HSA-Slit3 LRRD2 fusion protein (LRRD2-3).

5-1. Chemicals and Solvents

The carbamazepine used in this example was purchased from Sigma Aldrich, and HPLC grade acetonitrile and methanol were purchased from J. T. Baker.

5-2. Animals and Administration Conditions

In the present example, ICR-based male mice (6 weeks old, Orient Bio Co., Ltd., Seongnam, Republic of Korea) with a body weight ranging from 30 to 32.5 g were used. Mice were fasted for 4 hours before the experiment and fasting was maintained for up to 4 hours after administration. The breeding place was given 12 hours each of light and dark, and an appropriate temperature (20 to 25° C.) and humidity (40 to 60%) were maintained.

TABLE 4
Pharmacokinetic test

| Administered material | Number of animals | Administration dose |
|---|---|---|
| Slit3 LRRD2 | 4 | 10 mg/kg |
| HSA-Slit3 LRRD2 (LRRD2-3) | 3 | 35 mg/kg |
| Total | 7 | — |

Slit3 LRRD2 was prepared by being dissolved in PBS at a dose of 1 mg/mL. HSA-Slit3 LRRD2 (LRRD2-3) was prepared by being dissolved in PBS at a dose of 3.5 mg/mL (1 mg/mL for Slit3 LRRD2) in consideration of the molecular weight. The dose was 10 mL/kg in both groups, and the prepared solution was administered through the left caudal vein.

5-3. Pharmacokinetic Test

In the case of the pharmacokinetic test, fasted mice were administered Slit3 LRRD2 and HSA-Slit3 LRRD2 (LRRD2-3) at a dose of 10 mg/kg and 35 mg/kg, respectively, through the caudal vein. After administration, mice were fixed by hand at 0.05, 0.12, 0.33, 1, 3, 7, 10, 24, 48, and 72 hours, respectively, and then 70 μL of blood was collected from the right orbital venous plexus using heparin-coated capillary tubes. The collected blood was centrifuged for 5 minutes and then stored frozen at −20° C. until plasma was isolated and analyzed.

5-4. Analysis Method

The concentration of Slit3 LRRD2 in plasma samples was quantified using an HPLC/MS/MS system. Before sample pretreatment, plasma samples were purified using Ni-NTA magnetic beads. After purified Slit3 LRRD2 and HSA-Slit3 LRRD2 (LRRD2-3) were denatured by adding 6M urea and 18 mM dithiothreitol (DTT) thereto, alkylation was induced using 225 mM iodine acetamide. Then, to obtain a signature peptide, 850 ng of recombinant porcine trypsin (V5117, Promega, Madison, Wis., USA) was added thereto, and the resulting mixture was reacted in a water bath set at 37° C. for 24 hours. After 50 μL of 3% formic acid dissolved in MeOH was added to 70 μL of a trypsin digestion product produced after the reaction, the mixed sample was suspended using a vortex mixer for 10 minutes, centrifuged at 13,500 rpm for 10 minutes, and 160 μL of the supernatant was taken and transferred to an analysis vessel, and 5 μL of the transferred supernatant was injected into an HPLC MSMS system to perform analysis.

Detailed analysis conditions are as follows.
HPLC system: Agilent 1100 (Agilent Technologies, Santa Clara, Calif.)
Column: ZORBAX® C₈ 3.5 μm, 2.1*50 mm (Agilent)
Mobile phase:
   A: 0.1% formic acid dissolved in distilled water
   B: Acetonitrile
   (Isocratic elution)

| | |
|---|---|
| Time | 0 → 0.1 → 1.0 → 1.5 → 2.5 → 3 → 5 |
| B (%) | 5 → 5 → 5 → 95 → 95 → 5 → 5 |

Flow rate: 300 μL/min
Temperature: 20° C. in column, and 10° C. in autosampler tray
Runtime: 5 minutes
Detection: Tandem quadrupole mass spectrometer (API 4000, QTRAP®, Applied Biosystems/MDS SCIEX, Foster City, Calif., USA)
Curtain gas: 20 psi
Ion source gas 1: 50 psi
Ion source gas 2: 60 psi
Ionspray voltage: 5500 V
Temperature: 600° C.
Multiple-reaction-monitoring (MRM) mode: Positive

The molecular ions of a Silt3 LRRD2 signature peptide (P6) were fragmented by a collision energy of 23 V, and a collision gas was set to ‘medium (8 psi)’ in the equipment. Ions were detected in the ESI-positive MRM mode, and P6 was quantified from 587.97 to 491.50 in units of m/z. Detected peaks were integrated using Analyst software version 1.4.2 (Applied Biosystems/MDS SCIEX). A quantifiable range of Silt3 LRRD2 in plasma was 1 to 100 μg/mL, and that of HSA-Silt3 LRRD2 (LRRD2-3) was 3 to 100 μg/mL. In the corresponding analysis, Slit3 LRRD2 showed a peak retention time of 3.29 minutes.

5-5. Data Analysis

The concentration of CNC00000 in plasma over time was determined using the LC-MS/MS analysis method described in Example 5-4, and pharmacokinetic parameters (PK parameters) were calculated using non-compartmental analysis of WinNonlin® 4.2 (Pharsight Corp., Cary, N.C., USA) software. The maximum concentration (Cₘₐₓ) and the maximum concentration arrival time (Tₘₐₓ) were temporally calculated from a curve according to the blood drug concentration vs. time, and the elimination rate constant (Kₑ) was calculated by a linear regression analysis in the terminal phase of the log scale. The half-life (Ti/2) was calculated by dividing LN2 by Kₑ, and an area under the curve of blood drug concentration vs. time (AUC_{0-∞}) and an area under the curve of blood drug moment vs. time (AUMC_{0-∞}) were calculated by the linear trapezoidal rule and the standard area extrapolation method. Clearance (CL) and steady state volume of distribution (Vss) were calculated by the following [Equation 1] to [Equation 3]:

$$\begin{matrix}CL = \frac{Dose}{{AUC}_{0 - ∞}} & [ Equation 1 ] \\ {V}_{ss} - MRT \times CL & [ Equation 2 ] \\ MRT = \frac{{AUMC}_{0 - ∞}}{{AUC}_{0 - ∞}} & [ Equation 3 ]\end{matrix}$$

5-6. Results

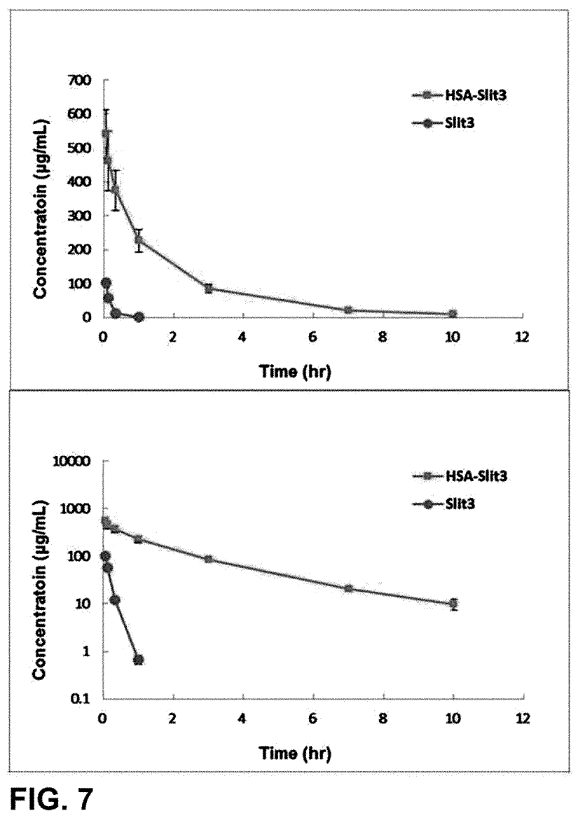

The concentrations of Slit3 LRRD2 and HSA-Slit3 LRRD2 (LRRD2-3) in plasma over time are shown in FIG. 7 and Tables 5 and 6, and pharmacokinetic parameters are shown in Table 6. The related parameters and all values were calculated for each individual and then averaged. Referring to the blood concentration pattern and animal experiment record over time, any abnormal populations were excluded from the data analysis, and the experimental group used for data analysis was set to have at least n=3 or more.

TABLE 5
Plasma concentration after intravenous administration of Slit3
Plasma concentration of Slit3 (μg/mL)

| Time (h) | #1 | #2 | #3 | #4 | mean | S.D. |
|---|---|---|---|---|---|---|
| 0.05 | 122 | 90.9 | 97.6 | 96.7 | 102 | 13.8 |
| 0.12 | 62.6 | 61.4 | 47.9 | 59.7 | 57.9 | 6.77 |
| 0.33 | 14.1 | 11.4 | 12.1 | 11.0 | 12.2 | 1.38 |
| 1 | 0.66 | 0.48 | 0.84 | 0.71 | 0.67 | 0.15 |
| 3 | BQL | BQL | BQL | BQL | 0.000 | — |
| 7 | BQL | BQL | BQL | BQL | 0.000 | — |
| 10 | BQL | BQL | BQL | BQL | 0.000 | — |
| 24 | BQL | BQL | BQL | BQL | 0.000 | — |

*BQL: When it is less than the quantification limit, it is treated as “0”.

TABLE 6
Plasma concentration after intravenous administration of HSA-Slit3
(LRRD2-3)
Plasma concentration of Slit3 (μg/mL)

| Time (h) | #6 | #7 | #8 | mean | S.D. |
|---|---|---|---|---|---|
| 0.05 | 587 | 460 | 577 | 541 | 70.6 |
| 0.12 | 539 | 366 | 480 | 462 | 87.9 |
| 0.33 | 355 | 327 | 441 | 374 | 59.4 |
| 1 | 217 | 199 | 262 | 226 | 32.4 |
| 3 | 82.8 | 73.6 | 98.9 | 85.1 | 12.8 |
| 7 | 18.9 | 18.5 | 23.4 | 20.3 | 2.72 |
| 10 | 9.71 | 7.64 | 12.5 | 9.95 | 2.44 |
| 24 | BQL | BQL | BQL | 0.000 | — |

*BQL: When it is less than the quantification limit, it is treated as ″0″.

As confirmed in the following Table 7, the HSA-Slit3 LRRD2 fusion protein (LRRD2-3) showed an approximately 14-fold improved half-life compared to Slit3 LRRD2.

TABLE 7

| | | | | HSA-Slit3 LRRD2 | | |
|---|---|---|---|---|---|---|
| | Slit3 LRRD2 | | | (LRRD2-3) | | |
| Parameter | Average | | S.D. | Average | | S.D. |
| Tₘₐₓ (h) | 0.050 | ± | 0.000 | 0.050 | ± | 0.000 |
| C₀ (μg/mL) | 153.9 | | 33.25 | 607.8 | | 59.87 |
| Cₘₐₓ (μg/mL) | 101.8 | ± | 13.79 | 541.3 | ± | 70.61 |
| T_{1/2} (h) | 0.139 | ± | 0.012 | 1.993 | ± | 0.147 |
| AUCₐₗₗ | 23.63 | ± | 2.534 | 919.9 | ± | 131.2 |
| (μg · h/mL) | | | | | | |
| AUC_{inf} | 23.77 | ± | 2.530 | 948.1 | ± | 136.1 |
| (μg · h/mL) | | | | | | |
| CL (mL/h/kg) | 424.0 | ± | 40.88 | 10.69 | ± | 1.504 |
| Vₛₛ (mL/kg) | 61.46 | ± | 7.368 | 24.1 | ± | 3.293 |

### Example 6

Confirmation of In Vivo Efficacy of HSA-Slit3 LRRD2 Fusion Protein

12-week-old SCID mice were treated with albumin-unbound Slit3 LRRD2 or the HSA-Slit3 LRRD2 fusion protein (LRRD2-3) for 4 weeks. Each drug was administered by intravenous injection once daily, five times per week, and Slit3 LRRD2 and the HSA-Slit3 LRRD2 fusion protein (LRRD2-3) were injected daily in a dose of 10 mg and 37.13 mg, respectively (Slit3 LRRD2 corresponds to 10 mg daily). A width of change was confirmed by measuring a bone mineral density for small animals before and after administration, and the results are shown in the following Table 8.

TABLE 8

| Group | Change in BMD (%) | | Change in BMC (%) | |
|---|---|---|---|---|
| Control (n = 12) | 5.27 ± | 1.10 | 9.51 ± | 2.51 |
| Slit3 LRRD2 (n = 12) | 6.94 ± | 0.83 | 10.91 ± | 2.44 |
| HSA-Slit3 LRRD2 | 9.03 ± | 1.18* | 18.78 ± | 2.87* |
| fusion protein | | | | |
| (LRRD2-3) (n = 11) | | | | |

*BMD: bone mineral density, BMC per unit area
*BMC: bone mineral content
*P < 0.05, vs. non-treated control

As shown in Table 8, albumin-unbound Slit3 LRRD2 has improved BMD and BMC, but their improvement was not statistically significant, and the HSA-Slit3 LRRD2 fusion protein (LRRD2-3) showed effects of significantly improving both BMD and BMC. Therefore, it was confirmed that the HSA-Slit3 LRRD2 fusion protein (LRRD2-3) exhibited a stronger therapeutic effect on bone-related diseases than albumin-unbound Slit3 LRRD2.

### INDUSTRIAL APPLICABILITY

Since the albumin-bound LRRD2 of the Slit3 protein exhibits the same cytological efficacy as albumin-unbound LRRD2 of the Slit3 protein and has a significantly increased in vivo half-life compared to albumin-unbound LRRD2 of the Slit3 protein, bone-related diseases can be more effectively prevented or treated.

The national research and development projects supporting the present invention are as follows.

(1) [National Research and Development Projects Supporting the Present Invention]

[Project Identification Number] 2017-1229 (HI15C0377010017)

[Ministry Name] Ministry of Health and Welfare

[Research Management Agency] Korea Health Industry Development Institute

[Research Project Name] Disease Oriented Translational Research

[Research Title] Discovery of macronuclear cell secretion factors with bone formation promotion

[Contribution Rate] 75/100

[Administrative Organization] Asan Medical Center, Seoul

[Research Period] Sep. 7, 2017 to Sep. 6, 2018

(2) [National Research and Development Projects Supporting the Present Invention]

[Project Identification Number] 2013-2234 (HI13C1634060018)

[Ministry Name] Ministry of Health and Welfare

[Research Management Agency] Korea Health Industry Development Institute

[Research Project Name] Disease Oriented Translational Research

[Research Title] Pharmacokinetic Study of Slit3 LRRD2 and in vivo Toxicity Verification Using Slit3 TG Mice

[Contribution Rate] 25/100

[Administrative Organization] Industry & Academic Cooperation in Chungnam National University (IAC)

[Research Period] Nov. 1, 2013 to Jun. 30, 2019

1. A method of preventing or treating bone-related diseases, comprising administering to a subject in need thereof a therapeutically effective amount of albumin-bound LRRD2 of the SLIT3 protein.

2. The method of claim 1, wherein the albumin is human serum albumin.

3. The method of claim 2, wherein the human serum albumin is bound to the N-terminus of the LRRD2 of the Slit3 protein.

4. The method of claim 3, wherein the human serum albumin comprises an amino acid sequence of SEQ ID NO: 2.

5. The method of claim 3, wherein the LRRD2 of the Slit3 protein comprises an amino acid sequence of SEQ ID NO: 3.

6. The method of claim 1, wherein the protein further comprises a linker between the albumin and the LRRD2 of the Slit3 protein.

7. The method of claim 6, wherein the linker is (GGGGS)n, wherein n is an integer from 1 to 10.

8. The method of claim 1, wherein the albumin-bound LRRD2 of the SLIT3 protein is administered as an injection.

9. The method of claim 1, wherein the bone-related disease is any one or more selected from the group consisting of osteoporosis, fractures, bone loss, osteoarthritis, metastatic bone cancer, and Paget's disease.


SEQUENCE LISTING (18 sequences)

SEQ ID NO: 1 - protein, 20 aa, Artificial
  SP Cystatin S
MARPLCTLLLLMATLAGALA

SEQ ID NO: 2 - protein, 585 aa, Artificial [sequence omitted]

SEQ ID NO: 3 - protein, 209 aa, Artificial
  Slit3 LRRD2
ISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCS

SEQ ID NO: 4 - protein, 8 aa, Artificial
  FLAG
DYKDDDDK

SEQ ID NO: 5 - protein, 5 aa, Artificial
  GS linker
  REPEAT (1)..(5): The amino acid residues at Position 1 to Postion 5 are repeated 1 to 10 times.
GGGGS

SEQ ID NO: 6 - protein, 15 aa, Artificial
  GS linker
GGGGSGGGGSGGGGS

SEQ ID NO: 7 - protein, 681 aa, Artificial
  LRRD2-1
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLLTSLVLYGNKITEIAKGLFDGLVSLQLLLLNANKINCLRVNTFQDLQNLNLLSLYDNKLQTISKGLFADYKDDDDK

SEQ ID NO: 8 - protein, 743 aa, Artificial
  LRRD2-2
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPDYKDDDDK

SEQ ID NO: 9 - protein, 822 aa, Artificial
  LRRD2-3
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSDYKDDDDK

SEQ ID NO: 10 - protein, 696 aa, Artificial
  LRRD2-4
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSLTSLVLYGNKITEIAKGLFDGLVSLQLLLLNANKINCLRVNTFQDLQNLNLLSLYDNKLQTISKGLFADYKDDDDK

SEQ ID NO: 11 - protein, 758 aa, Artificial
  LRRD2-5
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPDYKDDDDK

SEQ ID NO: 12 - protein, 837 aa, Artificial
  LRRD2-6
MARPLCTLLLLMATLAGALADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSDYKDDDDK

SEQ ID NO: 13 - protein, 696 aa, Artificial
  LRRD2-7
MARPLCTLLLLMATLAGALALTSLVLYGNKITEIAKGLFDGLVSLQLLLLNANKINCLRVNTFQDLQNLNLLSLYDNKLQTISKGLFADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 14 - protein, 758 aa, Artificial
  LRRD2-8
MARPLCTLLLLMATLAGALAIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 15 - protein, 837 aa, Artificial
  LRRD2-9
MARPLCTLLLLMATLAGALAISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 16 - protein, 711 aa, Artificial
  LRRD2-10
MARPLCTLLLLMATLAGALALTSLVLYGNKITEIAKGLFDGLVSLQLLLLNANKINCLRVNTFQDLQNLNLLSLYDNKLQTISKGLFAGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 17 - protein, 773 aa, Artificial
  LRRD2-11
MARPLCTLLLLMATLAGALAIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK

SEQ ID NO: 18 - protein, 852 aa, Artificial
  LRRD2-12
MARPLCTLLLLMATLAGALAISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDYKDDDDK